(19) United States
(12) Patent Application Publication
(10) Pub. No.: US 2025/0170033 A1
(43) Pub. Date: May 29, 2025

(54) Stable Dentrifice Compositions With High Sodium Bicarbonate Loading

(71) Applicant: Lubrizol Advanced Materials, Inc., Cleveland, OH (US)

(72) Inventors: Hong Luo, Copley, OH (US); Elena S. Draganoiu, Vaughan (CA); Christoph Schlueter, Rosenheim (DE)

(73) Assignee: Lubrizol Advanced Materials, Inc., Cleveland, OH (US)

(21) Appl. No.: 18/851,376

(22) PCT Filed: Oct. 7, 2022
(86) PCT No.: PCT/US2022/045983

§ 371 Date: Sep. 26, 2024

Related U.S. Application Data
(60) Provisional application No. 63/324,423, filed on Mar. 28, 2022.

(51) Int. Cl. A61K 8/19; A61K 8/21; A61K 8/73; A61K 8/81; A61Q 11/00

(52) U.S. Cl. CPC A61K 8/19; A61K 8/21; A61K 8/73; A61K 8/8176; A61Q 11/00; A61K 2800/28; A61K 2800/31

(57) ABSTRACT

The present technology generally relates to stable dentifrice compostions comprising high amounts of sodium bicarbonate. More specifically. the disclosed technology relates to stable dentifrice compositions comprising a) at least 40 wt. % sodium bicarbonate: b) at least one stannous ion anticaries component: c) a stabilizer component comprising i) a carboxyvinyl polymer and/or copolymer: ii) polyvinylpyrrolidone; and iii) xanthan gum; and d) at least one humectant component.

### TECHNOLOGICAL FIELD

The present technology generally relates to stable dentifrice compositions comprising high amounts of sodium bicarbonate. More specifically, the disclosed technology relates to stable dentifrice compositions comprising a) at least 40 wt. % sodium bicarbonate; b) at least one stannous ion anticaries component; c) a stabilizer component comprising i) a carboxyvinyl polymers and/or copolymers; ii) polyvinylpyrrolidone; and iii) xanthan gum; and d) at least one humectant component. In one aspect, the dentifrice compositions of the present technology are anhydrous.

### BACKGROUND

Many different dentifrice compositions are known for cleaning, whitening, and providing better gum health to preserve the teeth. Of these known dentifrices, many include a high content of water-insoluble abrasives which aid in removing plaque and stain build-up on the teeth. The use of bicarbonate salts (baking soda) as a dentifrice or the incorporation of such salts into dentifrice compositions is well known in the art of oral care. The addition of bicarbonate salts into dentifrices is beneficial for several reasons such as for providing good plaque removing capabilities. It is known that sodium bicarbonate penetrates the plaque layer, disrupting the sticky polysaccharide matrix and loosening the structural integrity of the biofilm, enhancing the physical removal of plaque. Additionally, sodium bicarbonate is well known for improving the whitening properties of dentifrices by removing stain build-up on the tooth surfaces. Importantly, bicarbonate salts provide a clean fresh feeling in the oral cavity after brushing and rinsing with water.

In general, sodium bicarbonate is a desirable abrasive for dentifrice compositions because sodium bicarbonate particles are relatively soft as compared to most conventional abrasive materials used in dentifrice compositions. The American Dental Association has advised that only a slight degree of abrasion is necessary to keep stains at bay on tooth enamel surfaces. However, due to its softness, high levels of sodium bicarbonate or a secondary abrasive are needed to achieve a maximum cleaning benefit.

However, high levels of conventional bicarbonate abrasives in toothpaste formulations impart excessively high viscosities, and make mixing difficult during the formulation of the product. The highly viscous nature of the product makes it difficult to dispense or extrude from the container by consumers. Morevover, as the product ages stability problems such as hardening or phase separation occur which is unacceptable.

In addition to formulating sodium bicarbonate abrasives into a dentifrice composition, it is known to formulate stannous containing compounds (e.g., stannous fluoride) into sodium bicarbonate containing dentifrices. Stannous fluoride is a common active ingredient in dentifrices and is recognized as an effective anticaries agent. Additionally, stannous fluoride is proven to be an effective agent for treating various conditions and diseases in the oral cavity including plaque, gingivitis, hypersensitivity, enamel decalcification, and periodontitis. Maintaining the amount of tin in the stannous state (tin II) is essential to providing these oral health benefits over the shelf-life of the product. However, stannous ions are unstable and rapidly oxidize and/or hydrolyze to the stannic ion (tin IV) which is not bioactive and also causes discoloring and staining issues. Moreover, stannic compounds are known to reduce or inhibit enamel fluoridation. Maximizing and maintaining the amount of tin in the stannous state (tin II) is essential to providing these oral health benefits over the shelf-life of the product.

Thus, stannous containing formulations typically include stabilization systems designed to maintain the stannous ion in the tin (II) state. The most effective approach to stabilize stannous compounds is to reduce or eliminate the amount of water present in the composition. However, reducing the level of water, and optionally replacing some or all of the removed water with an anhydrous humectant, may create problems in obtaining acceptable rheology in the composition and interfere with the physical stability of the product.

To date, attempts to achieve acceptable abrasive cleaning power and desirable anticaries efficacy by incorporating sodium bicarbonate at high levels as well as conventional amounts of stannous fluoride into a dentifrice composition have been unsatisfactory from a stability standpoint. It is known that the stabilizer component in a dentifrice is crucial to maintaining the physical stability of dentifrices such as toothpastes. Accordingly, an aspect of the present technology is the provision of a stabilizer component that stabilizes a dentifrice composition comprising high levels of sodium bicarbonate abrasive and a stannous containing anticaries agent.

### Summary of the Disclosed Technology

The present technology is based on the discovery that a stabilizer component comprising: i) a carboxyvinyl polymer; ii) polyvinylpyrrolidone; and iii) xanthan gum stabilizes a dentifrice composition containing high levels of sodium bicarbonate and a stannous containing anticaries agent against poor stability, dispensibility, texture consistency, and discoloring.

Another aspect of the present technology is the provision of a sodium bicarbonate based anhydrous toothpaste formulation containing stannous fluoride which is stabilized by a stabilizer component comprising i) a carboxyvinyl polymer; ii) polyvinylpyrrolidone; and iii) xanthan gum, which is effective in removing plaque and retarding stain build-up on the teeth without deleterious abrasion of tooth enamel, cementum or dentin, and wherein the toothpaste exhibits good physical stability and substantially overcomes formulation problems caused by high viscosities.

In one aspect, the present technology relates to a dentifrice composition comprising:
a) from about 40 to about 70 wt. %, or from about 45 to about 65 wt. %, or from about 50 to about 60 wt. % of an abrasive component comprising sodium bicarbonate;
b) from about 0.15 wt. % to about 2.5 wt. %, or from about 0.2 to about 2.45 wt. %, or from about 0.25 to about 2.4 wt. %, or from about 0.5 to about 2.25 wt. %, or from about 0.75 to about 2 wt. %, or from about 1 to about 1.5 wt. % of an anticaries component selected form a stannous ion source;
c) a stabilizer component comprising:
   i) from about 0.3 to about 1 wt. % of a carboxyvinyl polymer;
   ii) from about 0.2 to about 1 wt. % of polyvinylpyrrolidone;
   iii) from about 0.2 to about 1 wt % of xanthan gum; and
d) from about 15 to about 50 wt. %, or from about 20 to about 45 wt. %, or from about 25 to about 40 wt. % of at least one humectant component; wherein all weight percentages are based on the weight of the total composition.

### Detailed Description of the Disclosed Technology

In all aspects of the disclosed technology all percentages are calculated by the weight of the total composition. All ratios are expressed as weight ratios. All numerical ranges of amounts are inclusive and combinable unless otherwise specified.

While overlapping weight ranges for the various components and ingredients that can be contained in the disclosed compositions have been expressed for selected embodiments and aspects of the disclosed technology, the amount of each component in the disclosed compositions is selected from its disclosed range such that the sum of all components or ingredients in the composition will total 100 weight percent. The amounts employed will vary with the purpose and character of the desired product and can be readily determined by one skilled in the art.

All percentages, ratios, and levels of components referred to herein are based on the 100 percent active material, unless otherwise indicated.

The dentifrice compositions of the disclosed technology may suitably comprise, consist essentially of, or consist of, the components, elements, and process delineations described herein. The disclosed technology illustratively disclosed herein suitably may be practiced in the absence of any element which is not specifically disclosed herein.

As defined herein, “stable” and “stability” means that no visible phase separation is observed for a period of at least about one week of storage, or at least about 1 month of storage, or at least 3 months of storage under accelerated aging conditions at 40° C. ±2° C. at 75% ±5% relative humidity (RH). In another aspect, the products of the disclosed technology show no visible phase separation or syneresis after about at least 6 months of accelerated storage conditions at 40° C. ±2° C. at 75% RH ±5% RH and 12 months for long-term (LT) conditions at 25° C. ±2° C. at 60% R ±5% RH.

The prefix “(meth)acryl” includes “acryl” as well as “methacryl”. For example, the term “(meth)acrylic acid” includes both acrylic acid and methacrylic acid.

The term “dentifrice” as used herein means paste, gel, powder, tablets, or liquid formulations, unless otherwise specified, that are used to clean the surfaces of the oral cavity and may be in the form of toothpaste, tooth gel, tooth powders, tablets, foams, strips and rinses. In one aspect the dentifrice is a toothpaste or tooth gel.

The components of the dentifrice compositions of the present technology are described in the following paragraphs.

### Abrasive Component

In one aspect of the present technology, the dentifrices of the present technology employ sodium bicarbonate as an abrasive component. As previously recognized, sodium bicarbonate provides plaque removing capabilities and leaves a clean, fresh feeling in the mouth after brushing and rinsing with water. In one aspect, the sodium bicarbonate is provided in particulate form having a mean particle size ranging from 5μm to about 200μm, or from about 20μm to 120μm in diameter. The bicarbonate particles are incorporated in the dentifrice composition in an amount ranging from about 40 to about 70 wt. %, or from about 45 to about 65 wt. %, or from about 50 to about 60 wt. %, based on the weight of the total dentifrice composition.

### Stannous Ion Component

In one aspect, the dentifrice of the present technology includes a stannous ion. The stannous ion generally comes from a stannous salt that is added to the dentifrice. Stannous ions have been found to help in the reduction of gingivitis, plaque, sensitivity, and improved breath benefits. In one aspect, the stannous salt is selected from stannous fluoride and stannous chloride dihydrate. Other stannous salts include stannous acetate, stannous chlorofluoride, stannous pyrophosphate, stannous formate, stannous acetate, stannous gluconate, stannous lactate, stannous tartrate, stannous oxalate, stannous malonate stannous citrate, stannous ethylene glyoxide, and combinations thereof. Combinations of two or more of the aforementioned stannous salts may also be used. The stannous salts are present in an amount of from about 0.15 wt. % to about 2.5 wt. %, or from about 0.2 to about 2.45 wt. %, or from about 0.25 to about 2.4 wt. %, or from about 0.5 to about 2.25 wt. %, or from about 0.75 to about 2 wt. %, or from about 1 to about 1.5 wt. %, based on the weight of the total dentifrice composition.

### Stabilizer Component

In one aspect, the stabilizer component comprises: i) a carboxyvinyl polymer; ii) polyvinylpyrrolidone; and iii) xanthan gum. The stabilizer component improves the processibility of dentifrice compositions containing high levels of bicarbonate salts during manufacture by reducing the formulation viscosity during mixing and improves the long-term stability (shelf-life) of the end product against phase separation and hardening. Additionally, the stabilizer component stabilizes the stannous ion component against oxidation and/or hydrolysis to the stannic state thereby maintaining stannous ion efficacy for the oral health benefits previously described and mitigating enamel fluoridation inhibition, as well as illuminating staining of tooth enamel.

In one aspect, constituent i) of the stabilizer component is a carboxvinyl polymer. Examples of carboxyvinyl polymers include carbomers (e.g., polyacrylic acid homopolymers crosslinked with allyl pentaerythritol, allyl ether of sucrose or allyl ether of propylene), polycarbophils (e.g., polyacrylic acid homopolymers crosslinked with divinyl glycol), and crosslinked copolymers of (meth) acrylic acid and C₁₀ to C₃₀ alkyl esters of (meth) acrylic acid (e.g., copolymers crosslinked with allyl pentaerythritol, allyl ether of sucrose or allyl ether of propylene; INCI: Acrylates/C10-30 Alkyl Acrylate Crosspolymer), and crosslinked interpolymers of acrylic acid in optional combination with a C₁₀ to C₃₀ alkyl ester of (meth) acrylic acid. Interpolymers of crosslinked acrylic acid and crosslinked copolymers of (meth) acrylic acid and a C₁₀ to C₃₀ alkyl ester of (meth) acrylic acid are polymerized in the presence of a steric stabilizer and/or a wetting agent.

U.S. Pat. No. 5,288,814, which is incorporated herein by reference, describes crosslinked interpolymers of (meth) acrylic acid alone or in optional combination with comonomer(s) of a C₁₀ to C₃₀ alkyl ester of (meth) acrylic acid which are polymerized in the presence of a steric stabilizer comprising at least one hydrophilic moiety and at least one hydrophobic moiety arranged in a linear block copolymer configuration or a random comb copolymer configuration. Both of these steric stabilizer configurations contain hydrophilic moieties comprising polyoxyethylene ether groups. U.S. Pat. No. 5,373,044, which is herein incorporated by reference, discloses interpolymers of (meth) acrylic acid and optional comonomer(s) of a C₁₀ to C₃₀ alkyl ester of (meth) acrylic acid which are polymerized in the presence of the steric stabilizer described in U.S. Pat. No. 5,288,814 supra, and a wetting agent. The wetting agent is selected from a low surface tension surfactant, glycols, polyhydric alcohols, and mixtures thereof.

Suitable carbomers described above are commercially available under the Carbopol™ trade name marketed by Lubrizol Advanced Materials, Inc. under product designations 956, 971P NF, 974P NF and 980 NF.

Suitable polycarbophil polymers are marketed by Lubrizol Advanced Materials under the Noveon™ trade name, product designation AA-1 USP.

Copolymers of (meth) acrylic acid and C₁₀ to C₃₀ alkyl esters of (meth) acrylic acid are commercially available under the Pemulen™ trade name, product designations TR-1 NF and TR-2 NF, marketed by Lubrizol Advanced Materials, Inc.

Interpolymers of (meth) acrylic acid in optional combination with a C₁₀ to C₃₀ alkyl ester of (meth) acrylic acid are commercially available under the Carbopol™ trade name, product designations ETD 2020 NF and Ultrez 10 NF, marketed by Lubrizol Advanced Materials, Inc.

In one aspect of the present technology, constituent ii) of the stabilizer component comprises polyvinylpyrrolidone (PVP). Polyvinylpyrrolidone is a linear polymer obtained by radical polymerization of N-vinylpyrrolidone. Polyvinylpyrrolidone may be characterized by a K-value which is a viscosity index directly related to molecular weight. Commercially available polyvinylpyrrolidone is divided into several viscosity grades according to its K value, e.g., K-12, or K-17, or K-25, or K-29/32, or K-90, with a weight average molecular weight of 4,000; 10,000; 34,000; 58,000; and 1,300,000; respectively, as described in the Ashland Pharamaceutical and Technology Report entitled Plasticizer Compatibility and Thermal and Rheological Properties of Plasdone™ Povidone and Copovidone Polymer for Hot-Melt Extrusion Applications; Mohammed Rhaman, Seher Ozkan, James Lester, Ishrathe Farzana, Vivian Bi and Thomas Durig. K values are calculated values obtained from viscosity measurements which are plugged into the Fikentscher equation.

In one aspect, medium to high K values, e.g., K-25, K-29/32, K-90, and mixtures thereof are suitable for use as the polyvinylpyrrolidone constituent of the stabilizer component. Phamaceutical grade polyvinylpyrrolidone is commercially available from Ashland Global Specialties Inc., under the Plasdone™ trade name and from BASF Corporation under the Kollidon™ tradename.

In one aspect, constituent iii) of the stabilizer component of the present technology comprises xanthan gum. Xanthan gum is a natural gum polysaccharide commonly employed as a food additive and is also widely used in cosmetics and personal care industry as a rheology control agent. It is produced by a biotechnological process involving fermentation of glucose or sucrose by the bacterium, Xanthomonas campestris. Xanthan gum is a high molecular weight exocellular heteropolysaccharide ranging from about 1,000,000 dalton units to above about 50,000,000 dalton units per polymer molecule. Xanthan gum is commercially available from C.P. Kelco under the tradename Keldent™. Xanthan gum is also available from a number of other commercial sources including Sigma-Aldrich.

In one aspect, the stabilizer component comprises: i) from about 0.3 to about 1 wt. % of a carboxylvinyl polymer; ii) from about 0.2 to about 1 wt. % of polyvinylpyrrolidone; and iii) from about 0.2 to about 1 wt % of xanthan gum, based on the weight of the total composition.

### Humectants

In one aspect, suitable humectants for use in the present technology include polyhydric alcohols such as glycerine, sorbitol, xylitol, propylene glycol, polyethylene glycol (PEG) as well as other polyols and mixtures of these humectants.

In one aspect, low to medium molecular weight polyethylene glycol, e.g., PEG 300, PEG 400, PEG 600, PEG 800, PEG 1000, PEG 1500 and mixtures thereof are useful as the humectant component.

In one aspect, the humectant component is present in an amount ranging from about 15 to about 50 wt. %, or from about 20 to about 45 wt. %, or from about 25 to about 40 wt. %, based on the total weight of the composition.

### Auxiliary Dental Abrasives

Auxiliary dental abrasives may optionally be employed with the sodium bicarbonate abrasive of the present technology. The auxiliary abrasive selected must be one which is compatible with the sodium bicarbonate abrasive, as well as with the other components in the composition of interest and does not excessively abrade dentin. Suitable auxiliary abrasives include, for example, silicas including gels and precipitates; insoluble sodium polymetaphosphate; hydrated alumina; calcium containing compounds including calcium carbonate, sodium carbonate, sodium bicarbonate, dicalcium orthophosphate dihydrate, calcium pyrophosphate, tricalcium phosphate, calcium polymetaphosphate, calcium oxapatite; resinous abrasive materials such as particulate condensation products of urea and formaldehyde; and mixtures thereof.

In one aspect, the auxiliary dental abrasive is selected from calcium carbonate. Suitable sources include finely ground natural chalk (FGNC), ground calcium carbonate, precipitated calcium carbonate, and combinations thereof. Calcium carbonate abrasives are generally used in dentifrice compositions such as toothpastes that do not require a translucent to clear appearance, i.e., are opaque.

In one aspect the calcium carbonate is selected from FGNC obtained from limestone or marble. FGNC may also be modified chemically or physically by coating during milling or after milling by heat treatment. Typical coating materials include magnesium stearate or oleate. The morphology of FGNC may also be modified during the milling process by using different milling techniques, for example, ball milling, air-classifier milling or spiral jet milling. One example of natural chalk is described in International Patent Application Pub. No. WO 03/030850 having a medium particle size of 1 to 15 microns and a BET (Brunauer, Emmett and Teller) surface area of 0.5 to 3 m²/g.

In one aspect, the auxiliary dental abrasive is selected from an abrasive silica. Silica dental abrasives of various types are well-known in the art and possess the unique benefits of exceptional dental cleaning and polishing performance without unduly abrading tooth enamel or dentine.

The silica dental abrasives polishing materials herein, as well as other auxiliary abrasives, generally have an average particle size ranging between about 0.1 to about 30 microns, or from about 5 to about 20 microns. The abrasive can be precipitated silica or silica gels such as the silica xerogels described in U.S. Pat. No. 3,538,230 and U.S. Pat. No. 3,862,307. Commercially available examples include the silica xerogels marketed under the trade name Syloid™ by the W.R. Grace and Co. and precipitated silica abrasive materials marketed by Evonik Corporation under the trade name, Zeodent™ particularly the silicas carrying the designation Zeodent™ 103, Zeodent™ 113, Zeodent™ 115, Zeodent™ 124 and Zeodent™ 623. Another oral care abrasive silica from Evonik is marketed under the trade name Spherilex™ 145 and Spherilex™ 148. The types of silica dental abrasives useful in the toothpastes of the present technology are described in more detail in U.S. Pat. Nos. 4,340,583; 5,603,920; 5,589,160; 5,658,553; 5,651,958; and 6,740,311.

It is to be noted that there are two types of silica that can be used in toothpaste and tooth gel compositions. As discussed above silica is used as a dental abrasive. However, certain silicas may also be used as a thickener. The difference between the two types of silica is discussed in U.S. Pat. No. 6,342,205 (see Tables B and C and the accompanying disclosure), the disclosure of which is hereby incorporated by reference in its entirety. Generally, abrasive silicas have an oil absorption value ranging from about 50 to about 125 cm³/100 g, a pore volume ranging from about 1.5 to about 3 cm³/g, a pore density ranging from about 0.2 to about 0.3 g/cm^{3,} and a pack density ranging from about 0.35 to about 0.45 g/cm^{3.} In contrast, thickening silicas have higher oil absorption values, higher pore volumes, lower pore densities and lower pack densities than the abrasive silicas, wherein oil absorption value, pore volume, pore density and pack density is measured as set forth in columns 3 to 5 of U.S. Pat. No. 6,342,205 supra.

Mixtures of abrasives can be used such as, for example, mixtures of the various grades of Zeodent™ silica abrasives listed above. In one aspect, the total amount of auxiliary abrasive component in the dentifrice compositions of the present technology typically range from about 5 to about 30 wt. %, or from about 10 to about 25 wt. %, or from about 15 to about 20 wt. %, based on the total weight of the composition.

### Auxiliary Thickener

An optional auxiliary thickener provides a desirable consistency and/or enhances the performance of the dentifrice composition. The optional auxiliary thickener used in the compositions of the present technology is limited only to the extent that it may be added to a composition suitable for use in the oral cavity and provides desirable release of active components to the oral cavity. Suitable thickeners include cellulose derivatives (“cellulose gums”) such as carboxymethyl cellulose (CMC) and salts thereof (e.g., sodium CMC), methyl cellulose, hydroxyethyl cellulose, hydroxypropyl methyl cellulose, bacterial fermentation derived cellulose (FDC), microfibrous cellulose (MFC), and mixtures thereof; carrageenans such as iota-carrageenan, kappa-carrageenan, kappa-2-carrageenan, lambda-carrageenan, and mixtures thereof; guar gum; tara gum; locust bean gum, cassia gum, gum karaya; gum Arabic; gum tragacanth; and mixtures thereof; and silica.

FDC can be obtained from Sphingomonas ferment extract (commercially available as Kelco Care™ Diutan gum). Kelco Care™ Diutan gum is a natural high molecular weight polysaccharide with a low anionic charge density produced by fermentation of the mircrorganism, Sphingomonas sp. ATCC 53159. It is comprised of six sugar units of d-glucose, d-glucuronic acid, d-glucose (with 2 I-rhamnose in the side chain) and I-rhamnoses, forming a linear backbone with a repeating side chain. Diutan gum has high molecular weight (typically millions of kD) and thus long molecular chain length. This leads to Diutan gum polymer chain entanglement at relatively low concentrations in solution. The structured network of entangled, stiff molecules creates high viscosity at low shear rates, resulting in outstanding suspension properties. The molecules in the complex network of a Diutan gum formulation are weakly associated. This network is progressively disrupted under the influence of applied shear stress making diutan gum solutions highly pseudoplastic. This rheology behavior makes Sphingomonas ferment extract (Kelco Care™ Diutan gum) a robust candidate as a thickener and stabilizing agent in challenging dentifrice formulations such as low or high pH, high ion content or in natural formulations.

Microfibrous cellulose can be prepared by mechanically disrupting/altering cereal, wood, or cotton-based cellulose fibers, and is commercially available from various commercial sources.

As discussed previously, thickening silicas are differentiated over abrasive silicas by having higher oil absorption values, higher pore volumes, lower pore densities and lower pack densities than the abrasive silicas. In one aspect, thickening silicas have an oil absorption value ranging from about 150 to about 225 cm³/100 g, a pore volume ranging from about 3.5 to about 6.5 cm³/g, a pore density ranging from about 0.1 to about 0.15 g/cm^{3,} and a pack density ranging from about 0.1 to about 0.25 g/cm^{3.} As mentioned previously oil absorption values, pore volumes, pore densities and pack densities are measured as set forth in columns 3 to 5 of U.S. Pat. No. 6,342,205 supra. Silica thickener materials are marketed by Evonik Corporation under the trade name, Zeodent™, particularly the silicas carrying the designation Zeodent™ 153, Zeodent™ 163 and Zeodent™M 165.

In one aspect, the optional auxiliary thickener can be selected from the individual thickeners listed above, or mixtures of two or more thickeners selected from the list above can be utilized in the compositions of the present technology. In one aspect, the auxiliary thickener component is present in the dentifrice compositions of the present technology in an amount ranging from about 0.1 to about 10 wt. %, or from about 0.5 to about 8 wt. %, or about 0.75 to about 7.5 wt. %, or from about 1 to about 5 wt. %, based on the total weight of the composition.

### Surfactant

In one aspect, the dentifrice compositions of the present technology comprise at least one orally acceptable detersive surfactant. Surfactants enhance stability of a dentifrice composition, help clean the oral cavity surfaces through detergency, and provide foam upon agitation, e.g., during brushing with an oral care product of the disclosure, e.g., toothpastes and tooth gels. Surfactants facilitate the efficacy of dentifrice active components such as anticaries agents and whiteners by thoroughly dispersing these materials throughout the oral cavity. The detersive surfactant component can be selected from an anionic surfactant, an amphoteric surfactant, a nonionic surfactant, and mixtures thereof.

In one aspect, the surfactant may comprise an anionic surfactant. Suitable anionic surfactants include without limitation water-soluble salts of C₈-C₂₀ alkyl sulfates, sulfonated monoglycerides of C₈-C₂₀ fatty acids, sarcosinates, taurates, and the like. Illustrative examples include, but are not limited to sodium lauryl sulfate, sodium cocoyl monoglyceride sulfonate, sodium lauryl sarcosinate, sodium lauryl isoethionate, sodium laureth carboxylate, and sodium dodecyl benzenesulfonate.

In one aspect, the surfactant may comprise an amphoteric surfactant. Suitable amphoteric surfactants include without limitation alkyl betaines, e.g., lauryl betaine, coco betaine; alkylamido betaines, e.g., cocamidopropyl betaine and cocohexadecyl dimethylbetaine; alkylamido sultaines, e.g., cocamidopropyl hydroxysultaine; (mono-and di-) amphocarboxylates, e.g., sodium cocoamphoacetate, sodium lauroamphoacetate, sodium capryloamphoacetate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, C₈-C₂₂ alkyl amine oxides, e.g., octyldimethylamine oxide, decyldimethylamine oxide, dodecyldimethylamine oxide, iso-dodecyldimethyl amine oxide, myristyldimethylamine oxide, myristyl/cetyldimethylamine oxide, myristyldimethylamine oxide, cocodimethylamine oxide; and mixtures thereof.

In one aspect, the surfactant may comprise a nonionic surfactant. Suitable nonionic surfactants include without limitation poloxamers, polyoxyethylene sorbitan esters, polysorbates, fatty alcohol ethoxylates, alkylphenol ethoxylates, tertiary amine oxides, tertiary phosphine oxides, dialkyl sulfoxides and the like.

In one aspect, the surfactant may comprise a cationic surfactant. Suitable cationic surfactants include without limitation stearyldimenthylbenzyl ammonium chloride; dodecyltrimethylammonium chloride; nonylbenzylethyldimethyl ammonium nitrate; tetradecylpyridinium bromide; laurylpyridinium chloride; cetylpyridinium chloride; laurylpyridinium chloride; laurylisoquinolium bromide; ditallow (Hydrogenated) dimethyl ammonium chloride; dilauryldimethyl ammonium chloride; and stearalkonium chloride, and mixtures thereof.

In one aspect, the surfactant comprises an anionic surfactant, an amphoteric surfactant and a nonionic surfactant.

In one aspect the amount of surfactant present in the dentifrice compositions of the present technology range from about 0.1 to about 5 wt. %, or from about 0.25 to about 4.0 wt. %, or from about 0.4 to about 4 wt., or from about 1 to about 3 wt. %, based on the weight of the total composition.

### Fluoride Ion Source

The dentifrice composition of the present technology also may include a fluoride ion source to mitigate calcium loss. Suitable fluoride ion sources include without limitation sodium fluoride, potassium fluoride, barium fluoride, potassium monofluorophosphate, sodium monofluorophosphate, ammonium monofluorophosphate, sodium fluorosilicate, ammonium fluorosilicate, an amine fluoride such as Olaflur (N′-octadecyltrimethylendiamine-N,N, N′-tris (2-ethanol)-dihydrofluoride), ammonium fluoride, sodium fluorosilicate, ammonium fluorosilicate, sodium fluorozirconate, sodium monofluorophosphate, aluminum monofluorophosphate, aluminum difluorophosphate, and combinations thereof. In one aspect, one or more fluoride ion sources are included in the dentifrice composition.

In one aspect, the dentifrice composition of the present technology includes a fluoride ion source in an amount ranging from about 0.01 to about 10 wt. %, or from about 0.1 to about 7 wt. %, or from about 0.2 to about 5 wt. %, or from about 0.25 to about 1.75 wt. %, or from about 0.5 to about 1.5 wt. %, or from about 0.75 to about 1.25 wt. %, based on the total weight of the composition.

In one aspect, the amount of fluoride in the dentifrice composition can be expressed in terms of the active fluoride ion present in the composition. The active fluoride ion may be present in an amount ranging from about 500 to about 20,000 ppm, or from about 850 to about 15,000 ppm, or from about 1,500 to about 10,000 ppm, or from about 2,500 to about 8,000 ppm, or from about 3,000 to about 5,000 ppm.

### Water

Water is an optional component of the dentifrice compositions of the present technology. Water employed in the preparation of the dentifrice composition should preferably be deionized and free of organic impurities. In one aspect, the dentifrice composition comprises from about 0 to 50 wt. %, or from about 1 to about 45 wt. %, or from about 5 to about 40 wt. %, or from about 10 to about 35 wt. %, or from about 15 to about 25 wt. % water, wherein all percentages are based on the weight of the total composition. These ranges of free water are in addition to the water which is introduced with other materials in the composition.

### Other Components

In addition to the above described components, the compositions of the present technology can contain a variety of other conventional ingredients typically used in dentifrice compositions such as toothpastes and tooth gel compositions. Such components include, but are not limited to, flavoring agents, sweetening agents, sensates, pH adjusting agents and buffering agents, coloring agents, anticalculus agents, antibacterial agents, preservatives, whitening agents, desensitizing agents, and mixtures thereof.

In some aspects, the compositions of the present technology comprise at least one flavoring agent. Suitable flavoring agents include but are limited to essential oils, various flavoring aldehydes, esters, alcohols, and similar materials, as well as menthol, carvone, and anethole, as well as mixtures thereof. Examples of essential oils include oils of spearmint, peppermint, wintergreen, sassafras, clove, sage, eucalyptus, marjoram, cinnamon, lemon, lime, grapefruit, and orange. In some embodiments, a mixture of peppermint oil and spearmint oil is used as the flavorant in the compositions disclosed herein.

The amount of flavorant, if employed, ranges from about 0.1 to about 5 wt. %, or from about 0.2 to 4 wt. %, or from about 0.3 to about 3 wt. %, or from about 0.4 to about 2 wt., or from about 0.5 to 2 wt. %, or from about 0.6 to about 2 wt. %, or from about 0.7 to about 2 wt., or from about 0.8 to about 2 wt. %, or from about 0.9 to about 2 wt. %, or from about 1 to about 2 wt. %, based on the total weight of the composition.

In some aspects, the compositions of the present technology comprise at least one sweetening agent. Any food grade or pharmaceutically acceptable sweetener may be used. Suitable sweetening agents include, for example, sucrose, glucose, saccharin, sucralose, dextrose, levulose, lactose, mannitol, sorbitol, fructose, maltose, xylitol, saccharin salts (e.g., sodium saccharin), thaumatin, aspartame, D-tryptophan, dihydrochalcones, acesulfame, cyclamate salts, and mixtures thereof.

The amount of sweetening agent, if employed, ranges from about 0.005 to about 10 wt. %, or from about 0.01 to 9 wt. %, or from about 0.1 to 7 wt. %, or from about 0.1 to 5 weight %, or from about 0.3 to about 3 wt. %, or from about 0.5 to 2 wt. %, or from about 0.6 to about 1 wt. %, based on the total weight of the composition.

Sensates such as cooling, warming, and tingling agents are useful to deliver signals to the consumer. The most well-known cooling agent is menthol, particularly 1-menthol, which is found naturally in peppermint oil. Among synthetic cooling agents, many are derivatives of or are structurally related to menthol, i.e., containing the cyclohexane moiety, and derivatized with functional groups including carboxamide, ketal, ester, ether and alcohol. Examples include the p-menthanecarboxamide compounds such as N-ethyl-p-methan-3-carboxamide or N-(4-cyanomethylphenyl)-ρ-menthanecarboxamide. An example of a synthetic carboxamide cooling agent that is structurally unrelated to menthol is N,2,3-trimethyl-2-isopropylbutanamide. Additional exemplary synthetic cooling agents include alcohol derivatives such as 3-1-menthoxy-propane-1,2-diol, isopulegol, p-menthane-3,8-diol; menthone glycerol acetal; menthyl esters such as menthyl acetate, menthyl acetoacetate, menthyl lactate, and monomenthyl succinate.

Additional agents that are structurally unrelated to menthol but have been reported to have a similar physiological cooling effect include alpha-keto enamine derivatives described in United State U.S. Pat. No. 6,592,884, including 3-methyl-2-(1-pyrrolidinyl)-2-cyclopenten-1-one (3-MPC), 5-methyl-2-(1-pyrrolidinyl)-2-cyclopenten-1-one (5-MPC); 2,5-dimethyl-4-(1-pyrrolidinyl)-3 (2H)-furanone (DMPF); icilin (also known as AG-3-5, chemical name 142-hydroxyphenyl]-4-[2-nitrophenyl]-1,2,3,6-tetrahydropyrimidine-2-one).

Examples of warming agents include ethanol; nicotinate esters, such as benzyl nicotinate; polyhydric alcohols; nonanoyl vanillyl amide; nonanoic acid vanillyl ether; vanillyl alcohol alkyl ether derivatives such as vanillyl ethyl ether, vanillyl butyl ether, vanillyl pentyl ether, and vanillyl hexyl ether; isovanillyl alcohol alkyl ethers; ethylvanillyl alcohol alkyl ethers; veratryl alcohol derivatives; substituted benzyl alcohol derivatives; substituted benzyl alcohol alkyl ethers; vanillin propylene glycol acetal; ethylvanillin propylene glycol acetal; ginger extract; ginger oil; gingerol; zingerone; or combinations thereof.

Examples of tingling agents include capsaicin; homocapsaicin, jambu oleoresin, zanthoxylum peperitum, saanshool-I, saanshool II, sanshoamide, piperine, piperidine, spilanthol, 4-(1-methoxymethyl)-2-phenyl-1,3-dioxolane, or combinations thereof.

In one aspect, the amount of sensate ranges from about 0.001 to about 5 wt. %, or from about 0.01 to about 4 wt. %, or from about 0.1 to about 3 wt. %, or from about 0.5 to about 2 wt. %, or from about 1 to about 1.5 wt. %, based on the total weight of the composition.

The dentifrice compositions of the present technology may include an effective amount of a pH adjusting agent and/or a pH buffering agent. pH modifying agents, as used herein, refer to agents that can be used to adjust or maintain the pH of the dentifrice compositions to a desired pH range, based on formulation requirements. Such agents include acidifying agents to lower the pH, basifying agents to increase the pH and buffering agents to maintain the pH within a desired range. pH modifying agents and buffers may include alkali metal hydroxides, ammonium hydroxide, organic ammonium compounds, amino alcohols, carbonates, sesquicarbonates, borates, silicates, phosphates, imidazole, and mixtures thereof. Specific pH adjusting agents and buffers include monosodium phosphate (monobasic sodium phosphate), trisodium phosphate (sodium phosphate tribasic dodecahydrate or TSP), sodium benzoate, benzoic acid, sodium hydroxide, potassium hydroxide, alkali metal carbonate salts, sodium carbonate, imidazole, pyrophosphate salts, sodium gluconate, lactic acid, sodium lactate, citric acid, sodium citrate, tris (hydroxymethyl) aminomethane and/or phosphoric acid.

In one aspect the amount of pH adjusting agent and/or buffer utilized is an amount necessary to adjust and maintain the pH of the dentifrice compositions in a range from about pH 7.5 to about pH 10, or from about pH 7.5 to about pH 8.5, e.g., or from about pH 7.6 to about pH 8.4, or from about pH 7.7 to about pH 8.3, or a pH of about 8.

In some aspects, the dentifrice compositions may include at least one colorant. Colorants include pigments, dyes, lakes and agents imparting a luster or reflectivity such as pearling agents. Any orally acceptable colorant may be used, including without limitation talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and the like. Food, Drug and Cosmetic (FD&C) colorants such as primary FD&C Blue No. 1, FD&C Blue No. 2, FD&C Green No. 3, FD&C Yellow No. 5, FD&C Yellow No. 6, FD&C Red No. 3, FD&C Red No. 33 and FD& C Red No. 40 and lakes FD&C Blue No. 1, FD&C Blue No. 2, FD&C Yellow No. 5, FD&C Yellow No. 6, FD&C Red No. 2, FD&C Red No. 3, FD& C Red No. 33, FD&C Red No. 40 and combinations thereof.

The one or more colorants are optionally present in a total amount of from about 0.001 to about 20 wt. %, or from about 0.01 to about 10 wt. %, or from about 0.1 to about 5 wt. %, based on the total weight of the composition.

The dentifrice compositions may include an anticalculus agent. In one aspect, the anticalculus agent is a phosphorous containing compound. Non-limiting examples include a pyrophosphate salt as a source of pyrophosphate ion. In one aspect, the composition comprises sodium pyrophosphate (SPP), sodium acid pyrophosphate (SAPP), sodium tripolyphosphate (STPP), tetrasodium pyrophosphate (TSPP), sodium hexametaphosphate (SHMP), and combinations thereof. Other useful anticalculus agents include polycarboxylate polymers and polyvinyl methyl ether/maleic anhydride (PVME/MA) copolymers.

In one aspect, the amount of anticalculus agent ranges from about 0.01 to about 10 wt. %, or from about 0.1 to about 6 wt. % of the pyrophosphate salt, based on the weight of the total composition.

In one aspect, the compositions of the present technology can contain an antimicrobial (e.g., antibacterial) agent and/or a preservative. Antimicrobial agents and preservatives improve the antimicrobial characteristics of the dentifrice composition and improves storage life stability. Non-limiting examples of antibacterial and preservative agents are triclosan, chlorhexidine, cetylpyridinium chloride, benzalkonium chloride, stannous salts, essential oils, zinc oxide, zinc citrate, benzyl alcohol, sodium benzoate, isobutyl para-hydroxybenzoate, isopropyl para-hydroxybenzoate, ethyl para-hydroxybenzoate, methyl paraben, ethyl paraben, propyl paraben, and mixtures thereof. Other useful antibacterial agents are disclosed in U.S. Pat. No. 5,776,435.

The antibacterial and/or preservative agents are optionally present in an antimicrobial effective total amount, typically ranging from about 0.05 to about 10 wt. %, or from about 0.1 to about 3 wt. %, or from about 0.5 to about 1 wt. %, based on the total weight of the composition.

In one aspect, the dentifrice compositions of the present technology can contain a whitening agent. Non-limiting examples of whitening agents that may be used include, for example, peroxides, metal chlorites, perborates, percarbonates, peroxyacids, hypochlorites, and mixtures thereof. In some embodiments, the whitening agent is hydrogen peroxide or a hydrogen peroxide source, for example, urea peroxide or a peroxide salt or complex (for example, peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or a hydrogen peroxide polymer complex (for example, a peroxide-polyvinylpyrrolidone polymer complex).

In one aspect, the amount of whitening agent in the dentifrice compositions of the present technology ranges from about 1 to about 10 wt. %, or from about 0.25 to about 7.5 wt. %, or from about 0.4 to 5 wt. %, or from about 0.6 weight % to about 3 wt. %, based on the total weight of the composition.

In one aspect, the dentifrice compositions of the present technology optionally comprise a desensitizing, or tooth sensitivity protecting, agent. One or more such agents can be present. Suitable desensitizing agents include, without limitation, potassium salts such as potassium citrate, potassium tartrate, potassium chloride, potassium sulfate and potassium nitrate. Oher suitable desensitizing agents include sodium nitrate and stannous and strontium salts. Alternatively, a local or systemic analgesic such as aspirin, codeine, acetaminophen, sodium salicylate or triethanolamine salicylate can be used alone or in combination with the foregoing desensitizing agents.

In one aspect, the desensitizing agent is utilized in an amount ranging from about 0.1 to about 10 wt. %, or from about 0. 5 to about 7 wt. %, or from about 1 to about 5 wt. %, based on the total weight of the composition.

The dentifrice compositions of the present technology are prepared by conventional methods for making dentifrice formulations. In one aspect, the dentifrice is formulated into a toothpaste or tooth gel which can be prepared by the hot process or the ambient process (cold process). The process can be a batch process or continuous process. In one aspect, the dentifrice can be prepared by the process described in U.S. Pat. Nos. 4,353,890 and 6,187,293.

In one aspect, the dentifrice compositions of the present technology are manufactured following standard toothpaste formulation procedures well-known in the art. For example, the compositions can be manufactured as follows. Polymers are dispersed in pre-dispersing solvent (polyethylene glycol or propylene glycol or mixtures thereof) to obtain a pre-dispersed polymer mixture. The pre-dispersed polymer mixture is added into glycerin and placed in a planetary mixer (Ross VersaMix, Model VMC-1) and mixed under heat (approximately 50° C.) with full vacuum for 1-hour to obtain a homogeneous gel phase. The heating is stopped and salts like fluorides, sodium saccharin, phosphate salts, and pH adjuster are added into the gel phase and mixed under full vacuum for 30 minutes. Sodium bicarbonate and other solids are added into the mixture and mixed at a planetary speed of 20 to 30 rpm for 3 to 5 minutes until the bicarbonate is fully wetted. The mixture is then mixed at an increased planetary speed of 80 to 100 rpm and a disperser speed of 2500 to 3500 rpm under vacuum for 45 to 60 minutes to obtain a smooth dentifrice. Surfactants and flavoring agents are then added to the dentifrice and mixed under vacuum for 20 to 30 minutes with a planetary speed of 80 to 100 rpm and a disperser speed of 1500 to 2500 rpm to obtain a homomgeneous composition.

In toothpaste or gel form, the composition can be packaged in a conventional plastic laminated metal tube, a pump dispenser, a squeezable plastic container, and other conventional toothpaste and tooth gel dispensers. The toothpaste and gel formulations can be conveniently dispensed in ribbon-like form with good body and texture. The formulation does not overly sag over into the bristles of a toothbrush and is not lumpy or overly tacky.

The toothpaste and tooth gel compositions of the present technology are used in conventional manner. The compositions are brushed onto dental surfaces and subsequently rinsed away. The compositions generally contact the dental surfaces in a brushing motion for at least about 30, or at least about 60, or at least about 120 seconds.

The present technology is exemplified by the following examples that are merely for the purpose of illustration and are not to be regarded as limiting the scope of the technology or the way it can be practiced. Unless otherwise specified all weight percentages are expressed as 100 percent active material.

### EXAMPLES 1 TO 6

Dentifrice compositions were prepared from the components set forth in Table 1. All compositions contain 0.45% stannous fluoride and 50% sodium bicarbonate and manufactured under 4 kg scale by using a Ross mixer (Model # VMC-1). Carboxyvinyl polymer and polyvinylpyrrolidone (PVP) K30 were dispersed separately in polyethylene glycol, and xanthan gum was dispersed in glycerin to obtain individual pre-dispersed polymer mixtures. The pre-dispersed polymer mixtures and the rest of glycerin from the formulation were added into Ross mixer and mixed under heat (approximately 50° C.) under full vacuum for 1-hour to obtain a homogeneous gel phase. The heating was stopped and salts like fluorides, sodium saccharin, phosphate salts, and pH adjuster were added into the gel phase and mixed with a planetary speed of 80 to 100 rpm and a disperser speed of 2500 to 3500 rpm under full vacuum for 30 minutes. Sodium bicarbonate and other solids were added into the mixture and were mixed at an increased planetary speed of 80 to 100 rpm and a disperser speed of 2500-3500 rpm under vacuum for 45 to 60 minutes to obtain a smooth dentifrice. Surfactants and flavoring agents were added to the dentifrice and mixed under vacuum for 20 to 30 minutes until homogeneous. Three dentifrice compositions (Examples 1 to 3), were prepared with a stabilizer comprising carboxyvinyl polymer in combination with PVP and xanthan gum. Three comparative dentifrice compositions (Examples 4 to 6) were prepared with xanthan, PVP or xanthan/PVP stabilizer systems, respectfully, in the absence of the carboxyvinyl polymer.

TABLE 1

| Ingredients | | | | | | |
|---|---|---|---|---|---|---|
| (% w/w) | Ex. 1 | Ex. 2 | Ex. 3 | Ex. 4¹ | Ex. 5¹ | Ex. 6¹ |
| Formulation | Stannous | Stannous | Stannous | Stannous Fluoride | Stannous Fluoride | Stannous Fluoride |
| | Fluoride | Fluoride | Fluoride | Toothpaste | Toothpaste | Toothpaste |
| | Toothpaste | Toothpaste | Toothpaste | without | without | without |
| | with | with | with | Carboxyvinyl | Carboxyvinyl | Carboxyvinyl |
| | Stabilizer | Stabilizer | Stabilizer | Polymer | Polymer | Polymer |
| Sodium | 50.0 | 50.0 | 50.0 | 50.0 | 50.0 | 50.0 |
| Bicarbonate | | | | | | |
| Stannous fluoride | 0.45 | 0.45 | 0.45 | 0.45 | 0.45 | 0.45 |

Stabilizer

| | | | | | | |
|---|---|---|---|---|---|---|
| Carboxyvinyl | 0.3 | 0.4 | 0.5 | — | — | — |
| Polymer | | | | | | |
| Polyvinyl- | 0.3 | 0.4 | 0.5 | — | 1 | 0.5 |
| pyrrolidone | | | | | | |
| (PVP) | | | | | | |
| Xanthan | 0.3 | 0.4 | 0.2 | 1 | — | 0.5 |

Anhydrous Humectants:

| | | | | | | |
|---|---|---|---|---|---|---|
| Glycerin | 20.65 | 20.35 | 20.96 | 21.05 | 21.05 | 21.06 |
| PEG 400 | 15.0 | 15.0 | 15.0 | 15.0 | 15.0 | 15.0 |

Other Oral Care Ingredients:

| | | | | | | |
|---|---|---|---|---|---|---|
| Tetrasodium | 5 | 5 | 5 | 5 | 5 | 5 |
| Pyrophosphate | | | | | | |
| Thickening Grade | 5 | 5 | 5 | 5 | 5 | 5 |
| Silica | | | | | | |
| Surfactant³ | 1.5 | 1.5 | 1.5 | 1.5 | 1.5 | 1.5 |
| Tris(Hydroxy- | 0.5 | 0.5 | 0.5 | — | — | — |
| methyl) | | | | | | |
| Aminomethane | | | | | | |
| Flavorants, | 1 | 1 | 1 | 1 | 1 | 1 |
| Sweeteners, and | | | | | | |
| Colorants | | | | | | |

¹Comparative
²Carbopol ® 974P NF polymer
³Sodium Lauryl Sulfate

The toothpaste samples set forth in Table 1, along with two commercial anhydrous sodium bicarbonate toothpastes, were packaged in 8-oz. glass jars and stored inside an ESPEC humidity chamber (Model No. ESL-3CA) at 40±2° C. and 75% ±5 RH for an accelerated stability test (ACC Test), in accordance with the International Council for Harmonization of Technical Requirements for Pharmaceuticals for Human Use (ICH), (ICH Harmonised Tripartite Guideline Stability Testing Of New Drug Substances and Products Q1A (R2), Current Step 4, version dated 6 Feb. 2003), and at 25±2° C. and 60±5% RH for a long-term stability test (LT Test) in accordance with the ICH protocol, supra. The toothpaste viscosity (helipath viscosity was measured on a Brookfield viscometer (Model No. DV-I+) at 5 rpm and ambient room temperature of (20-25° C.)) was measured once every month for three months per ISO 11609: 2017 (E) stability test protocol and recorded (Table 2). Toothpaste performance and visual appearance observations, (syneresis/separation and discoloration) are summarized in Table 2.

TABLE 2

| Samples | A¹ | B¹ | Ex. 1 | Ex. 2 | Ex. 3 | Ex. 4 | Ex. 5 | Ex. 6 |
|---|---|---|---|---|---|---|---|---|
| pH (25% solids) | 8.18 | 8.17 | 8.59 | 8.69 | 8.73 | 8.58 | 8.53 | 8.62 |
| Helipath | 319,000 | 335,200 | 286,600 | 382,200 | 399,800 | 193,000 | 304,400 | 248,200 |
| Viscosity | | | | | | | | |
| (cP) | | | | | | | | |
| (Initial) | | | | | | | | |
| Helipath | 625,000 | 711,60 0 | 357,000 | 340,000 | 477,400 | 288,600 | 316,800 | 327,000 |
| Viscosity | | | | | | | | |
| (cP) | | | | | | | | |
| (1 month) | | | | | | | | |
| (ACC) | | | | | | | | |
| Helipath | 442,000 | 605,800 | 295,800 | 277,000 | 412,800 | 183,200 | 282,000 | 254,400 |
| Viscosity | | | | | | | | |
| (cP) | | | | | | | | |
| (1 month) (LT) | | | | | | | | |
| Appearance | ACC | ACC & LT | ACC & LT: | ACC & LT: | ACC & LT: | ACC & LT: | ACC & LT: | ACC & LT: |
| (1 month) | Syneresis & | Stable | Stable; | Stable; | Stable; | No | No | No |
| | Hardening; | | Smooth and | Smooth and | Smooth and | Syneresis; | Syneresis; | Syneresis; |
| | LT: Stable | | Glossy | Glossy | Glossy | Discolored | Discolored | Discolored |
| Melipath | 1,120,000 | 1,612,000 | 349,800 | 365,200 | 597,200 | 451,400 | 371,000 | 351,200 |
| Viscosity | | | | | | | | |
| (CP) | | | | | | | | |
| (2 months) | | | | | | | | |
| (ACC) | | | | | | | | |
| Helipath | 425,000 | 644,000 | 285,000 | 315,800 | 471,200 | 220,200 | 311,400 | 274,200 |
| Viscosity | | | | | | | | |
| (CP) | | | | | | | | |
| (2 months) | | | | | | | | |
| (LT) | | | | | | | | |
| Appearance | ACC and | ACC: | ACC and | ACC and | ACC and | ACC and | ACC and | ACC: |
| (2 months) | LT: Phase | Syneresis | LT: Stable; | LT: Stable; | LT; Stable; | LT: No | LT: No | Phase |
| | Separation | and | Smooth and | Smooth and | Smooth and | Syneresis; | Syneresis; | Separation; |
| | | Hardening; | Glossy | Glossy | Glossy | Discolored | Discolored | Discolored |
| | | LT: Stable | | | | | | |
| Helipath | — | — | 367,800 | 395,800 | 511,000 | 422,200 | 335,400 | — |
| Viscosity | | | | | | | | |
| (cP) | | | | | | | | |
| (3 months) | | | | | | | | |
| (ACC) | | | | | | | | |
| Helipath | — | — | 307,000 | 396,000 | 404,400 | 222,220 | 291,000 | — |
| Viscosity | | | | | | | | |
| (cP) | | | | | | | | |
| (3 months) | | | | | | | | |
| (LT) | | | | | | | | |
| Appearance | — | — | ACG & LT: | ACC & LT: | ACC & LT: | Failed | Failed | — |
| (3 months) | | | Stable; | Stable; | Stable; | Syneresis | Syneresis | |
| | | | Smooth and | Smooth and | Smooth and | and | and | |
| | | | Glossy | Glossy | Glossy | Discolored | Discolored | |

¹Commercially available anhydrous toothpaste containing 40-60 wt. % sodium bicarbonate (based on total weight of the composition)

The two commercial anhydrous sodium bicarbonate toothpaste (A) and (B) failed the stability test under ACC and LT test conditions and exhibited severe syneresis and hardening (poor dispensability/extrudability). All commercial samples had approximately 100% viscosity increase tested under the accelerated test conditions even at the first month. It is known that approximately 100% viscosity variation indicates the tested formulation has tendency to fail the stability test and gives unacceptable or poor physical stability test results.

Three compositions (Examples 1, 2, and 3), prepared with the stabilizer of the present technology, exhibited good physical stability and no sign for syneresis/separation, hardening and discoloration (yellowing effect) when aged for 3 months under accelerated storage conditions in accordance with the ACC and LT tests. Three comparative dentifrice compositions (Examples 4, 5, and 6), employing only two components of the stabilizer composition (PVP and xanthan) in the absence of the carboxyvinyl polymer component exhibited syneresis under accelerated aging conditions, and also had a tendency to discolor (severe yellow appearance on the surface), which is an indication of the oxidation of stannous ion from tin (II) form to its in-active form tin (IV).

1. A dentifrice composition comprising:
a) from about 40 to about 70 wt. %, or from about 45 to about 65 wt. %, or from about 50 to about 60 wt. % of an abrasive component comprising sodium bicarbonate;
b) from about 0.15 wt. % to about 2.5 wt. %, or from about 0.2 to about 2.45 wt. %, or from about 0.25 to about 2.4 wt. %, or from about 0.5 to about 2.25 wt. %, or from about 0.75 to about 2 wt. %, or from about 1 to about 1.5 wt. % of an anticaries component selected from a stannous ion source;
c) a stabilizer component comprising:
i) from about 0.3 to about 1 wt. % of a carboxyvinyl polymer;
ii) from about 0.2 to about 1 wt. % of polyvinylpyrrolidone;
iii) from about 0.2 to about 1 wt % of xanthan gum; and
d) from about 15 to about 50 wt. %, or from about 20 to about 45 wt. %, or from about 25 to about 40 wt. % of at least one humectant component; wherein all percentages are based on the weight of the total composition.

2. A dentifrice composition of claim 1, wherein said stannous ion source is selected from stannous fluoride, stannous chloride dihydrate, stannous chlorofluoride, stannous pyrophosphate, stannous sulfate, stannous formate, stannous acetate, stannous gluconate, stannous lactate, stannous tartrate, stannous oxalate, stannous malonate, stannous citrate, stannous ethylene glyoxide, and combinations thereof.

3. A dentifrice composition of claim 1 further comprising a fluoride ion source.

4. A dentifrice composition of claim 3, wherein said fluoride ion source is selected from sodium fluoride, potassium fluoride, barium fluoride, potassium monofluorophosphate, sodium monofluorophosphate, ammonium monofluorophosphate, sodium fluorosilicate, ammonium fluorosilicate, an amine fluoride such as Olaflur (N′-octadecyltrimethylendiamine-N,N,N′-tris (2-ethanol)-dihydrofluoride), ammonium fluoride, sodium fluorosilicate, ammonium fluorosilicate, sodium fluorozirconate, sodium monofluorophosphate, aluminum monofluorophosphate, aluminum difluorophosphate, and combinations thereof.

5. A dentifrice composition of claim 3, wherein said fluoride ion source is present in an amount ranging from about 0.01 to about 10 wt. %, or from about 0.1 to about 7 wt. %, or from about 0.2 to about 5 wt. %, or from about 0.25 to about 1.75 wt. %, or from about 0.5 to about 1.5 wt. %, or from about 0.75 to about 1.25 wt. %, based on the total weight of the composition.

6. A dentifrice composition of claim 1, wherein said carboxyvinyl polymer is selected from a crosslinked homopolymer of acrylic acid, a crosslinked copolymer of (meth) acrylic acid and a C₁₀-C₃₀ alkyl ester of (meth) acrylic acid, interpolymers thereof, and combinations thereof.

7. A dentifrice composition of claim 6, wherein said crosslinked copolymer of (meth) acrylic acid is prepared from acrylic acid and a C₁₀ to C₃₀ alkyl ester of acrylic acid.

8. A dentifrice composition of claim 1, wherein said at least one anhydrous humectant component is selected from glycerol, sorbitol, xylitol, polyethylene glycol, propylene glycol, and combinations thereof.

9. A dentifrice composition of claim 1, wherein said abrasive component comprises an auxiliary abrasive selected from abrasive silica, tricalcium phosphate (Ca₃(PO₄)₂), dicalcium phosphate dihydrate (CaHPO₄·2H₂O), hydroxyapatite (Ca₁₀(PO₄)₆ (OH)_{2,} calcium carbonate abrasive; or abrasives such as sodium metaphosphate, potassium metaphosphate, aluminum silicate, calcined alumina, bentonite, and combinations thereof.

10. A dentifrice composition of claim 9, wherein said auxiliary abrasive component is present in an amount ranging from about 5 to about 30 wt. %, or from about 10 to about 25 wt. %, or from about 15 to about 20 wt. %, based on the total weight of the composition.

11. A dentifrice composition of claim 1, further comprising a surfactant selected from an anionic surfactant, cationic surfactant, zwitterionic surfactant, amphoteric surfactant, nonionic surfactant, and mixtures thereof.

12. A dentifrice composition of claim 11, wherein said surfactant is present in an amount ranging from about 0.1 to about 5 wt. %, or from about 0.25 to about 4.0 wt. %, or from about 0.4 to about 4 wt., or from about 1 to about 3 wt. %, based on the weight of the total composition.

13. A dentifrice composition of claim 1, further comprising a thickener component selected from cellulosic thickeners, non-xanthan gums, seaweed extracts, thickening silica, and mixtures thereof.

14. A dentifrice composition of claim 13, wherein said cellulosic thickener is selected from carboxymethyl cellulose, sodium carboxymethyl cellulose, hydroxypropyl cellulose, methyl cellulose, hydroxyethyl cellulose, hydroxypropyl cellulose, hydroxypropyl methyl cellulose, bacterial fermentation derived cellulose, microfibrous cellulose, and mixtures thereof; or wherein said non-xanthan gum thickener is selected from guar gum, tara gum, locust bean, gum Arabic, gum tragacanth, karaya gum, cassia gum, and mixtures thereof; or wherein said seaweed extract thickener is selected from kappa-carrageenan, kappa-2-carrageenan, iota-carrageenan, lambda-carrageenan, alginates, and mixtures thereof.

15. A dentifrice composition of any of claim 13, wherein said thickener is present in an amount ranging from about 0.1 to about 10 wt. %, or from about 1.5 to about 9 wt. %, or about 2.5 to about 8.5 wt. %, or from about 4 to about 7.5 wt. %, based on the weight of the total composition.

16. A dentifrice composition of clam 1, further comprising a component selected from flavoring agents, sweetening agents, sensates, pH adjusting agents and buffering agents, coloring agents, anticalculus agents, antibacterial agents, preservatives, whitening agents, desensitizing agents, and combinations thereof.

17. A dentifrice composition of claim 1, wherein said composition is anhydrous.

18. A dentifrice composition of claim 1, wherein the dentifrice is a toothpaste or a tooth gel.

19-23. (canceled)
